(19) United States
(12) Patent Application Publication
(10) Pub. No.: US 2014/0271845 A1
(43) Pub. Date: Sep. 18, 2014

(54) COATED SUBSTRATE COMPOSITIONS

(71) Applicant: Cerovene, Inc., Valley Cottage, NY (US)

(72) Inventors: Manish S. SHAH, West Caldwell, NJ (US); Ray J. DiFalco, Ridgewood, NJ (US)

(73) Assignee: Cerovene, Inc., Valley Cottage, NY (US)

(21) Appl. No.: 14/218,383

(22) Filed: Mar. 18, 2014

Related U.S. Application Data
(60) Provisional application No. 61/792,988, filed on Mar. 15, 2013.

(51) Int. Cl. A61K 9/48; A61K 31/65; A61K 31/454; A61K 31/473; A61K 31/415; A61K 31/216; A61K 31/4439

(52) U.S. Cl. CPC A61K 9/4891; A61K 31/216; A61K 31/65; A61K 31/4439; A61K 31/473; A61K 31/415; A61K 31/454; A61K 9/4808
USPC 424/463; 514/543; 514/152; 514/338; 514/284; 514/406; 514/323

(57) ABSTRACT

Pharmaceutical compositions in unit dose form comprising a hollow substrate and one or more coatings on the hollow substrate, wherein at least one coating comprises at least one active pharmaceutical ingredient, and methods of making the same are provided.

### CROSS-REFERENCE TO RELATED APPLICATION

This application claims the benefit of U.S. Patent Provisional Application No. 61/792,988 filed on Mar. 15, 2013, which is incorporated herein by reference.

### FIELD OF THE INVENTION

The present invention relates, generally, to pharmaceutical compositions in unit dose form comprising a hollow substrate and one or more coatings on the hollow substrate, wherein at least one coating comprises at least one active pharmaceutical ingredient, and methods of making the same.

### DESCRIPTION OF THE RELATED ART

The formulation of drugs into capsules, such as soft or hard gelatin capsules, provides a number of benefits and has been known to solve many problems associated with tableting.

In a typical conventional capsule the pharmaceutical active ingredient is present inside the capsule. The typical method of producing such conventional pharmaceutical capsule, a pharmaceutical active ingredient is mixed together with diluents such as lactose and other ingredients such as solubilizers, antioxidants, chelating agents, buffers, emulsifiers, thickening agents, dispersants, and preservatives and the mixture is then filled into hard gelatin capsules. However, some problems are known to arise with these conventional capsules. For example, hard capsules are standardized in their size and volume, and there can be technical limitations with respect to active pharmaceutical ingredients (APIs) that are to be dosed in large quantities or very small quantities. It may be difficult to achieve a homogenous mixture of drug and excipient with a uniform amount of drug present in each capsule, and a small absolute variation in the percentage of the active ingredient in the capsule can correspond to a significant variation in the dose contained in each capsule, which is clearly most undesirable. Further, manufacturing of these capsules may be expensive if more than one dosage strength of the drug needs to be made, because the drug products having multiple strengths will have different fill weights and thus require capsules of multiple different sizes. Corresponding capsule machine change parts are needed to fill the corresponding capsule size. In addition, with many drugs, there are limitations on the amount of solubilizers and surfactants that are needed to achieve the desired characteristics, such as improved bioavailability. In addition, there are sometimes problems associated with conventional capsules after administrating to patients, especially in the presence of a food, due to physiological variability relating to, for example, intrinsic properties of the active pharmaceutical ingredients.

There are several currently marketed capsule products which are filled with small spherical particles or pellets, which are coated with active pharmaceutical ingredients. One such example is Antara® Capsules, which are filled with pellets coated with fenofibrate. Other example is Oracea® Capsules, which are filled with immediate-release and delayed release pellets of doxycycline. Prilosec® Capsules are filled with delayed release pellets of omeprazole. The process of manufacturing such drug-coated pellets typically requires fluid bed technology and several coating steps to achieve the desired potency of the pellets. The coated pellets are then sieved to achieve a narrow particle size distribution. Otherwise, they produce higher weight variation during encapsulation, which is not desirable. Overall, such processes are generally relatively more expensive. The limitation with respect to the encapsulation process is same as the as the conventional capsules as mentioned earlier.

U.S. Pat. No. 7,153,538 discloses methods of coating a pharmaceutical substrate with an active coating material, where the active coating material is preferably applied electrostatically. U.S. Pat. No. 7,153,538 also discloses that conventional spray coating techniques, such as the tumble coating method, are not appropriate for use where accuracy in the amount of the active material applied to the cores is required because there is little control over the amount of coating material applied to each core.

U.S. Pat. No. 4,670,287 discloses embodiments in which a drug-filled hard capsule is selectively coated with an enteric coating agent.

U.S. Pat. No. 6,350,468 discloses a double capsule where an internal capsule is placed inside an external one, and wherein each internal and external capsule includes one or more APIs.

U.S. Pat. No. 5,641,512 discloses an analgesic soft gelatin capsule, wherein a xanthine derivative, such as caffeine, is embedded in the capsule shell itself.

U.S. Patent Application Publication No. 20070212411 discloses coated hard and soft capsules containing at least one first drug in the capsule and at least a second drug in the coating.

U.S. Patent Application Publication 20100291201 discloses coated capsules containing a fill comprising inert ingredients and a coating comprising an active ingredient.

Japanese Patent Application Publication No. JP 59-157018 discloses capsules filled with an edible oil having a medicinal effect and coated with a powder having a medicinal effect.

All references cited herein are hereby incorporated by reference in their entirety.

### SUMMARY OF THE INVENTION

The present invention is generally directed to a pharmaceutical composition in unit dose form comprising a hollow substrate and one or more coatings on the hollow substrate, wherein at least one coating comprises at least one active pharmaceutical ingredient (API). In some embodiments, the hollow substrate comprises an empty hard or soft capsule.

Known capsule formulations typically contain a fill in the capsule shell, wherein the fill contains an active pharmaceutical ingredient and one or more pharmaceutically acceptable excipients. However, unlike these known formulations, embodiments of the present invention are directed to hollow substrates. In the present invention, at least one active pharmaceutical ingredient is present in the one or more coatings on the hollow substrate.

In some embodiments, additional coating(s) on the hollow substrate, such as immediate release coatings, protective coatings, enteric or delayed release coatings, sustained release coatings, barrier coatings, and combinations thereof may be placed between the hollow substrate and the at least one coating comprising the at least one API. In some embodiments, the hollow substrate may be coated with at least one top coating on the at least one coating comprising the at least one API, and may include, but are not limited to, immediate release coatings, protective coatings, enteric or delayed release coatings, sustained release coatings, barrier coatings, and combinations thereof.

One or more of the APIs of the present invention may also be formulated with a combination of one or more inactive ingredients including, but not limited to, solubilizers, antioxidants, chelating agents, buffers, emulsifiers, thickening agents, dispersants, and preservatives.

### DETAILED DESCRIPTION OF THE INVENTION

The present invention is directed to pharmaceutical compositions in unit dose form comprising a hollow substrate and one or more coatings on the hollow substrate, wherein at least one coating comprises at least one API. The composition is suitable for oral administration.

The term “hollow substrate” refers to a form suitable for oral administration comprising an empty cavity or space. The term “empty” means holding or containing no material or substantially no material. In some embodiments, the hollow substrate is a capsule, such as a hard capsule or soft capsule, which does not contain a capsule fill. The term “hollow substrate” includes forms that may contain minimal amounts of materials which are remnants from the manufacture of the form or which have otherwise been unintentionally formed within the form. For example, in embodiments wherein the substrate is a capsule, the term “hollow substrate” includes a capsule which may contain within the capsule shell any remnant materials, such as pieces of capsule shell that may have be contained therein during the manufacture of the capsule shell or during handling of the capsule shell.

The manufacture of substrates such as hard or soft capsules is generally known by those of ordinary skill in the art. For example, soft capsules may be made by various processes including the plate process, the rotary die process, the reciprocating die process, and the continuous process. See, for example, Ebert (1978), “Soft Elastic Gelatin Capsules: A Unique Dosage Form,” Pharmaceutical Technology 1(5); Reich (2004), “Chapter 11: Formulation and physical properties of soft capsules,” Pharmaceutical Capsules, 2d Ed., Pharmaceutical Press, 201-212, hereby incorporated by reference in their entireties. See also, U.S. Pat. No. 5,478,508 and U.S. Pat. No. 5,882,680, incorporated by references herein in their entireties, disclosing methods of manufacturing seamless capsules. Examples of the capsular materials include, but are not limited to, natural or synthetic gelatin, pectin, casein, collagen, protein, modified starch, polyvinyl pyrrolidone, acrylic polymers, cellulose derivatives (such as, but not limited to, hydroxypropyl methylcellulose (HPMC)), and combinations thereof, optionally with one or more plasticizers and/or water. Capsular materials may also include one or more preservatives, coloring and opacifying agents, flavorings and sweeteners, sugars, gastroresistant substances, or combinations thereof.

The shape and size of the capsules can vary in accordance with the invention. The shape of the capsule may be, but is not limited to, round, oval, tubular, oblong, twist off, or a non-standard shape (e.g., a fish, tree, star, heart, or bear), preferably oblong. The size of the capsule used will vary in accordance to the volume of the fill composition intended to be contained therein.

For example, in some embodiments of the present invention, hard or soft gelatin capsules may be manufactured in accordance with conventional methods as a single body unit comprising the standard capsule shape. A single-body soft gelatin capsule typically may be provided, for example, in sizes from 3 to 22 minims (1 minimum being equal to 0.0616 ml) and in shapes of oval, oblong or others. The gelatin capsule may also be manufactured in accordance with conventional methods, for example, as a two-piece hard gelatin capsule, sealed or unsealed, typically in standard shape and various standard sizes, conventionally designated as (000), (00), (0), (1), (2), (3), (4), and (5). The largest number corresponds to the smallest size.

In the present invention, one or more coatings of the pharmaceutical composition comprise one or more active pharmaceutical ingredients, or APIs.

The term “active pharmaceutical ingredient,” or API, includes any compound or drug which has pharmacological or biological activity.

In some embodiments, APIs include, but are not limited to, the following: analgesics, anti-inflammatory agents, anti-helminthics, anti-arrhythmic agents, anti-asthma agents, anti-bacterial agents, anti-viral agents, anti-coagulants, anti-dementia agents, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, immunosuppressants, anti-protozoal agents, anti-thyroid agents, anti-tussives, anxiolytics, sedatives, hypnotics, neuroleptics, neuroprotective agents, β-blockers, cardic inotropic agents, cell adhesion inhibitors, corticosteroids, cytokine receptor activity modulators, diuretics, anti-Parkinson's agents, gastro-intestinal agents, histamine H-receptor antagonists, keratolytics, lipid regulating agents, muscle relaxants, nitrates and other anti-anginal agents, non-steroid anti-asthma agents, nutritional agents, opioid analgesics, sex hormones, stimulants and anti-erectile dysfunction agents.

In some embodiments, the API comprises an antibiotic such as doxycycline (free base) or a salt thereof. Salts of doxycycline include, but are not limited to, hyclate (hydrochloride), calcium, monohydrate, carrageenate, phosphate, and mono-sodium-tetraphosphate salts of doxycycline. In some embodiments, the API comprises a fibrate such as fenofibrate or a salt thereof, such as choline fenofibrate. In some embodiments, the API comprises a proton pump inhibitor such as omeprazole or a salt thereof. Salts of omeprazole include, but are not limited to, lithium, sodium, magnesium, and calcium salts of omeprazole. In some embodiments, the API comprises a prostate drug such as dutasteride or a salt or solvate thereof. In some embodiments, the API comprises an anti-inflammatory drug such as celecoxib or a salt thereof. Salts of celecoxib include, but are not limited to, lithium, sodium, magnesium, and calcium salts of celecoxib. In some embodiments, the API comprises a cancer drug such as thalidomide or a salt thereof.

The pharmaceutical composition may comprise one or more pharmaceutically acceptable excipients. Examples of pharmaceutically acceptable excipients include, but are not limited to the following: anti-adhesives, inert fillers/diluents/binders, lipophilic agents and pigments. Other suitable pharmaceutically acceptable excipients are described in Remington: The Science and Practice of Pharmacy, Lippincott Williams and Wilkins, Baltimore, Md. (1995), incorporated herein by reference.

Fillers/diluents/binders may be incorporated such as sucrose, sorbitol, mannitol, various grades of lactose, various grades of microcrystalline cellulose, dextrins, maltodextrins, starches or modified starches, sodium phosphate, calcium phosphate, calcium carbonate, gelatin, polyvinylpyrrolidone, and sodium carboxymethylcellulose.

Disintegrants may be used such as cellulose derivatives, including microcrystalline cellulose, low-substituted hydroxypropyl cellulose, croscarmellose sodium, alginic acid, insoluble polyvinlypyrrolidone, and sodium carboxymethyl starch.

Glidants and lubricants may be incorporated such as stearic acid, metallic stearates, talc, waxes, and glycerides with high melting temperatures, colloidal silica, sodium stearyl fumarate, polyethyleneglycols, and alkyl sulphates.

Surfactants may be employed such as non-ionic (various grades of polysorbate); anionic such as docusate sodium and sodium lauryl sulfate, and cationic such as benzalkonium chloride. An example of an amphoteric surfactant is 1,2-diacyl-L-phosphatidylcholine. The preferred surfactants are TWEEN® 80, BRIJ®, and Nanoxyl-100.

Other appropriate pharmaceutically acceptable excipients may include colorants, flavoring agents, pH adjusting agents, solubilizing agents, wetting agents, solvent resistant agents and buffering agents.

One or more pharmaceutically acceptable excipients, may be added to any or all of the one or more coatings, provided that they do not interfere with the drug and provide a desired benefit to the pharmaceutical. In preferred embodiments, the pharmaceutically acceptable excipients enhance the effect of the drug.

In some embodiments, the pharmaceutically acceptable excipients may enhance the activity of the active pharmaceutical ingredient, such as by increasing bioavailability, augmenting the effect of, increasing the Cmax, decreasing the Tmax, or otherwise benefically affecting the activity of the active pharmaceutical ingredient. In some embodiments, pH adjusting agents (e.g., basifying agents or acidifying agents) such as sodium bicarbonate, calcium carbonate and tartaric acid can be used to adjust the pH in the body and increase absorption of pH-sensitive active pharmaceutical ingredients.

The one or more coatings or layers on the hollow substrate may be applied by any coating technique including those described in Remington: The Science and Practice of Pharmacy, 22^{nd} Edition, 2012, Pharmaceutical Press, and 21^{st} Edition, 2005, (Lippincott Williams & Wilkins). Coating processes include coating using equipment including, but are not limited to, a standard or conventional coating pan, a Pelligrini pan system or other enclosable coating system, an immersion sword system, an immersion tube system, a perforated coating pan, and a fluidized bed. The coating may be applied by conventional coating or electrostatic coating. With standard pan coating, the coating material is typically applied by spraying the material on a rotating capsule bed, and heated air is directed into the pan and onto the tablet bed surface. The Pelligrini coating pan system includes a somewhat angular pan that rotates on a horizontal axis, and it generally involves the use of a diffuser to distribute drying air over the capsule bed. The Pelligrini system, like some other systems, is enclosable, in that they are capable of having one or more openings closed off. Immersion sword systems, such as the Glatt immersion-sword system, incorporate the use of perforated metal sword device that is immersed in the capsule bed, and drying air flows upward from the sword through the capsule bed. Immersion tube systems involve the use of a tube immersed in the capsule bed. The tube typically includes a spray nozzle built in the tip, and the tube also delivers heated air. Perforated coating pans include a perforated or partially performated drum that is rotated on its horizontal axis in an enclosed housing. Examples of perforated coating systems include the Accela-cota and Hi-coater systems, which involve the use of drying air that is directed into the drum, passed through the capsule bed, and passed through perforations in the drum. Other examples of perforated coating systems include the Driacoater system and the Glatt coater systems. In fluidized bed coating, a gas (generally air) is typically passed through a quantity of solids present in a holding vessel from the bottom. The solids are intensely dispersed, extending the surface of the solids to the air. During fluidized bed coating, fluidized particles are continuously sprayed with a coating liquid. In some embodiments, the one or more coatings are applied by spray coating.

The coating(s) may be applied, for example, as a solution, suspension, spray, dust or powder. In some embodiments, the coating solution, suspension, or spray comprises a solvent, and the coating is therefore solvent-based. Examples of solvents include, but are not limited to ethanol, methanol, acetone, methyl salicylate, water, and mixtures thereof. In some embodiments, the coating liquid (solution, suspension) may comprise solvent blends, such as chloroform-ethanol blends, ethyl lactate-ethanol blends, methyl salicylate-ethanol blends, toluene-ethanol blends, and methylene chloride-ethanol blends.

The present invention provides that at least one coating applied to the outside of the hollow substrate comprises an API. In some embodiments the thickness of this layer is from 5-800 microns, preferably 10-600 microns, more preferably 20-400 microns, most preferably 40-200 microns. In some embodiments, this layer is expressed in terms of percentage weight gain, based on the total weight of the hollow substrate including any layers provided on the hollow substrate prior to the at least one coating comprising the API. This layer may have a weight gain of 0.05-80%, preferably 0.1-60%, more preferably 1-50%, and most preferably 5-20%.

Some embodiments of the present invention provide that the at least one coating comprising the API includes an amount of at least one compound sufficient to improve the solubility of the at least one active pharmaceutical ingredient for a pharmaceutically acceptable duration of time. In some embodiments, the at least one compound comprises at least one polymer. The amount of polymer(s) to the amount of the API is preferably from about 1:20 to about 20:1 by weight, preferably from 1:5 to about 10:1 by weight. In embodiments where the amount of API is less than about 15 mg, the amount of polymer(s) is preferably from about 1:2 to about 5:1, and more preferably from about 1:1 to about 4:1. In embodiments where the amount of API is about 20 mg or more, the amount of polymer(s) is preferably about 1:4 to about 4:1, and more preferably about 1:3 to about 2:1. The polymers may include any pharmaceutically acceptable polymers known to those of skill in the art. Preferred polymers include, but are not limited to, cellulose derivatives such as hydroxyethyl cellulose, hydroxypropyl cellulose, hydroxypropyl methylcellulose, polyvinylpyrrolidone, polyvinylpyrrolidone/vinyl acetate copolymer, ethyl cellulose aqueous dispersions and combinations thereof, preferably hydroxpropyl cellulose, ethyl cellulose, and mixtures thereof. The preferred polymers may also include one or more of the polymers disclosed throughout the application or mixtures thereof.

In some embodiments of the present invention, the API is provided in a coating solution or suspension which is applied to the hollow substrate. In preferred embodiments, the API is provided in a homogenous coating solution or a heterologous suspension in a pharmaceutically acceptable solvent, preferably an aqueous or organic solvent. Pharmaceutically acceptable organic solvents have the advantages that they may be evaporated or sublimated during production, do not deform, melt, or otherwise change the structure of the hollow substrate (e.g., gelatin in a soft gelatin capsule), and do not generally cause agglomeration of the coated hollow substrates. In preferred embodiments, the pharmaceutically acceptable organic solvent is selected from methanol, ethanol, isopropranol, ethylene glycol, acetone, or mixtures thereof.

Additional pharmaceutically acceptable organic solvents that may be used include, but are not limited to, polypropylene glycol; polypropylene glycol; polyethylene glycol (for example, polyethylene glycol 600, polyethylene glycol 900, polyethylene glycol 540, polyethylene glycol 1450, polyethylene glycol 6000, polyethylene glycol 8000 (all available from Union Carbide), and the like); pharmaceutically acceptable alcohols which are liquids at about room temperature (for example, propylene glycol, ethanol, 2-(2-ethoxyethoxy)ethanol (TRANSCUTOL™, Gattefosse, Westwood, N.J. 07675), benzyl alcohol, glycerol, polyethylene glycol 200, polyethylene glycol 300, polyethylene glycol 400 and the like); polyoxyethylene castor oil derivatives (for example, polyoxyethyleneglycerol triricinoleate or polyoxyl 35 castor oil (CREMOPHOR™ EL, BASF Corp.), polyoxyethyleneglycerol oxystearate (CREMOPHOR™ RH 40 (polyethyleneglycol 40 hydrogenated castor oil) or CREMOPHOR™ RH 60 (polyethyleneglycol 60 hydrogenated castor oil), BASF Corp.), and the like); saturated polyglycolized glycerides (for example, GELUCIRE™ 35/10, GELUCIRE™ 44/14, GELUCIRE™ 46/07, GELUCIRE™ 50/13 or GELUCIRE™ 53/10 and the like, available from Gattefosse, Westwood, N.J.); polyoxyethylene alkyl ethers (for example, cetomacrogol 1000 and the like); polyoxyethylene stearates (for example, PEG-6 stearate, PEG-8 stearate, polyoxyl 40 stearate NF, polyoxyethyl 50 stearate NF, PEG-12 stearate, PEG-20 stearate, PEG-100 stearate, PEG-12 distearate, PEG-32 distearate, PEG-150 distearate and the like); ethyl oleate, isopropyl palmitate, isopropyl myristate and the like; dimethyl isosorbide; N-methylpyrrolidinone; parafin; cholesterol; lecithin; suppository bases; pharmaceutically acceptable waxes (for example, carnauba wax, yellow wax, white wax, microcrystalline wax, emulsifying wax and the like); pharmaceutically acceptable silicon fluids; soribitan fatty acid esters (including sorbitan laurate, sorbitan oleate, sorbitan palmitate, sorbitan stearate and the like); pharmaceutically acceptable saturated fats or pharmaceutically acceptable saturated oils (for example, hydrogenated castor oil (glyceryl-tris-12-hydroxystearate), cetyl esters wax (a mixture of primarily C₁₄-C₁₈ saturated esters of C₁₄-C₁₈ saturated fatty acids having a melting range of about 43-47° C.), glyceryl monostearate; and the like.

The coatings may also include a coating material, such as a film forming material and/or binder, and optionally other conventional additives such as lubricants, surfactants, fillers and antiadherents. Preferred coating materials may include antioxidants, buffers, solubilizers, dyes, chelating agents, disintegrants, and/or absorption enhancers. Surfactants may act as both solubilizers and absorption enhancers. The coating(s) may be formulated for immediate release, delayed or enteric release, or sustained release of the API in accordance with methods well known in the art. Conventional coating techniques are described, e.g., in Remington's Pharmaceutical Sciences, 18th Ed. (1990), hereby incorporated by reference.

Additional coatings to be employed in accordance with the invention may include, but are not limited to, for example, one or more immediate release coatings, protective coatings, enteric or delayed release coatings, sustained release coatings, barrier coatings, and combinations thereof.

An immediate release coating is coating which can rapidly release the drug from the dosage form. Rapid breakdown of the film in gastric media is important, leading to effective disintegration and dissolution. Eudragit RD100 (Rohm) is an example of such a coating. It is a combination of a water insoluble cationic methacrylate copolymer and a water soluble cellulose ether. In powder form, it is readily dispensable into an easily sprayable suspension that dries to leave a smooth film. Such films rapidly disintegrate in aqueous media at a rate that is independent of pH and film thickness.

A protective coating layer (i.e., seal coat) may be applied, if desired, by conventional coating techniques such as pan coating or fluid bed coating using solutions of polymers in water or suitable organic solvents or by using aqueous polymer dispersions. Suitable materials for the protective layer include cellulose derivatives such as hydroxyethyl cellulose, hydroxypropyl cellulose, hydroxypropyl methylcellulose, polyvinylpyrrolidone, polyvinylpyrrolidone/vinyl acetate copolymer, ethyl cellulose aqueous dispersions and the like. The protective coating layer may include antioxidants, chelating agents, colors or dyes. One of the functions of the protective coating is that it can stabilize the drug when it is exposed to accelerated conditions of temperature and humidity. The protective coating may also provide alcohol resistance to the dosage form and thus help to prevent dose dumping of the drug.

A delayed release or enteric coating layer may be applied onto the hollow substrate itself, or onto other coatings on the hollow substrate, with or without seal coating, by conventional coating techniques, such as pan coating or fluid bed coating using solutions of polymers in water or suitable organic solvents or by using aqueous polymer dispersions. All commercially available pH-sensitive polymers are included. Typically in such uses, the API is not released in the acidic stomach environment of approximately below pH 4.5, but not limited to this value. The pharmaceutical active should become available when the pH-sensitive layer dissolves at the greater pH; after a certain delayed time; or after the unit passes through the stomach. If utilized, the preferred delay time is in the range of two to six hours.

Delayed release or enteric polymers include cellulose acetate phthalate, cellulose acetate trimellitate, hydroxypropyl methylcellulose phthalate, polyvinyl acetate phthalate, carboxymethylethylcellulose, co-polymerized methacrylic acid/methacrylic acid methyl esters such as, for instance, materials known under the trade name EUDRAGIT® L12.5, L100, or EUDRAGIT® S12.5, S100 or similar compounds used to obtain enteric coatings. Aqueous colloidal polymer dispersions or re-dispersions can be also applied, e.g. EUDRAGIT® L 30D-55, EUDRAGIT® L100-55, EUDRAGIT® S100, EUDRAGIT® preparation 4110D (Rohm Pharma); AQUATERIC®, AQUACOAT® CPD 30 (FMC); KOLLICOAT® MAE 30D and 30DP (BASE); EASTACRYL® 30D (Eastman Chemical).

A sustained release film coat may include, but is not limited to, a water insoluble material such as a wax or a wax-like substance, fatty alcohols, shellac, zein, hydrogenated vegetable oils, water insoluble celluloses, polymers of acrylic and/or methacrylic acid, and any other slowly digestible or dispersible solids known in the art. The solvent for the hydrophobic coating material may be organic or aqueous. Preferably, the hydrophobic polymer is selected from (i) a water insoluble cellulosic polymer, such as an alkylcellulose, preferably ethylcellulose; (ii) an acrylic polymer; or (iii) mixtures thereof. In other preferred embodiments of the present invention, the hydrophobic material comprising the controlled release coating is an acrylic polymer. Any acrylic polymer which is pharmaceutically acceptable can be used for the purposes of the present invention. The acrylic polymers may be cationic, anionic or non-ionic polymers and may be acrylates, methacrylates, formed of methacrylic acid or methacrylic acid esters. Examples of suitable acrylic polymers include but are not limited to acrylic acid and methacrylic acid copolymers, methacrylic acid copolymers, methyl methacrylate copolymers, ethoxyethyl methacrylates, cynaoethyl methacrylate, methyl methacrylate, copolymers, methacrylic acid copolymers, methyl methacrylate copolymers, methyl methacrylate copolymers, methyl methacrylate copolymers, methacrylic acid copolymer, aminoalkyl methacrylate copolymer, methacrylic acid copolymers, methyl methacrylate copolymers, poly(acrylic acid), poly(methacrylic acid, methacrylic acid alkylamine copolymer, poly(methyl methacrylate), poly(methacrylic acid) (anhydride), methyl methacrylate, polymethacrylate, methyl methacrylate copolymer, poly(methyl methacrylate), poly(methyl methacrylate) copolymer, polyacrylamide, aminoalkyl methacrylate copolymer, poly(methacrylic acid anhydride), and glycidyl methacrylate copolymers.

A barrier coat may be included between the hollow substrate and an outer coat, between outer coats, or on the outermost coat. The barrier coat may be comprised of an enteric or delayed release coat (as above) or a barrier (non-functional) layer, which serves as a protective coat and/or scavenger to prevent leaching from the hollow substrate (e.g., glycerol or water) to the outer API component or vice versa. For example, in some embodiments a barrier coat may be used to prevent leaching of glycerol and/or water inside the hollow substrate into the API.

Embodiments of the invention may also include one or more coatings on the hollow substrate comprising one or more sequestrants, such as but not limited to, citric acid, citric acid monohydrate, dibasic sodium phosphate, phosphoric acid, potassium citrate, sodium citrate dihydrate, and the like, and/or one or more scavengers, such as but not limited to, salts or polymers preferably having ester and/or carboxylic acid groups, as known to those of skill in the art.

In some embodiments, the dosage form may be provided with a lag time between the administration of a first portion of API in one coating and the administration of second portion of API in another coating, e.g., by a delayed release or enteric coating provided as a barrier layer. In other embodiments, there is an immediate release of the first portion of the API, followed by a delayed or sustained release of the second (and/or further) portion of the API. In further embodiments, there is a delayed release of the first portion, followed by a bolus of the second (and/or further) portion.

Some preferred embodiments have at least one top coating on the coating comprising the at least one API, selected from the group consisting of immediate release coatings, protective coatings, enteric or delayed release coatings, sustained release coatings, barrier coatings, and combinations thereof.

As noted above, polymeric coatings are generally applied as aqueous-based solutions, organic-based solutions or dispersions, in which polymer-containing droplets are atomized with air or an inert gas and sprayed onto the substrate. Heated air or an inert gas may be added to the coating equipment to facilitate evaporation of the solvent and film formation. In the case of soft gelatin capsules, the processing parameters of spray rate and bed temperature must be controlled. Because gelatin is soluble in water, spraying an aqueous-based polymeric material at a high rate could lead to solubilization of the gelatin and capsule agglomeration. A high bed temperature may result in the evaporation of residual water from the capsule shell, causing the capsule to become brittle. Therefore, embodiments of the present invention comprises a method of coating soft gelatin capsules in which these consequences are avoided.

In addition, the deposition of the API onto the surface of the hollow substrate with high degree of accuracy could be affected by several factors. The accuracy of deposition needs to be demonstrated by evaluating coating uniformity which includes the mass variance of the coated hollow substrate and the variance of the content of the coated API.

In general, “uniformity of dosage unit” is defined as the degree of uniformity in the amount of the drug substance among dosage units (i.e., capsules). The uniformity of dosage unit can be demonstrated by, for example, the content uniformity method or the weight variation method, as appropriate. For example, the content uniformity method is based upon an assay of the individual content of drug substance(s) in a number of individual dosage units to determine whether the individual content is within the limits set. See, for example, USP 30 <905>“Uniformity of Dosage Units” pages 378-382, which is incorporated by reference herein in its entirety. In embodiments of the present invention, content uniformity of an active ingredient (i.e., either or both of the first API and the API, preferably at least the API) is within about 15% or less of the intended dosage, preferably within about 10% or less of the intended dosage, and more preferably within about 6% or less of the intended dosage. Content uniformity of an active ingredient is preferably controlled within a factor of about 15% or less between capsules, more preferably within a factor of about 10% or less, and even more preferably within a factor of about 6% or less between capsules.

Embodiments of the present invention provide for a method of coating a hollow substrate with at least one coating comprising an API, the method comprising controlling the rate of coating deposition on the hollow substrate and controlling the temperature during the coating process to produce a physically and chemically stable coated hollow substrate. This method also allows for a content uniformity of the API within a factor of about 15% or less of the intended dose, preferably about 6% or less of the intended dose.

Other embodiments of the present invention provide for a method of administering a coated hollow substrate in accordance with the invention to a subject for treatment of any of the diseases or conditions for which the API(s) may be used. For example, when the API comprises a lipid regulation agent, the method of administration may include treatment of at least one condition or disease independently selected from the group consisting of hypertriglyceridemia, hypercholesterolemia, mixed dyslipidemia, coronary heart disease (CHD), vascular disease, atherosclerotic disease and related conditions. The method of administration can also include treatment of other conditions or diseases such as, but not limited to, infections, gastrointestinal conditions, genitourinary conditions, pain or inflammation-related conditions, and cancer.

### Example 1

### Composition of a Capsule Dosage Form as Per the Present Invention

| Item # | Ingredients | Mg |
|---|---|---|
| 1 | Hollow empty HPMC Capsules Size # 2 | 60.00 |

Sugar-coating Step

| | | |
|---|---|---|
| 2 | Sucrose | 30.0 |
| 3 | Gum Acacia | 3.0 |
| 4 | Talc | 5.0 |
| 5 | Purified water | Qs |
| Total | | 98.0 |

### Example 2

### Drug Layering Formula

| | | |
|---|---|---|
| 1 | Hollow substrate from Example 1 | 98.0 |
| 2 | Purified water, USP | — |
| 3 | Hydroxypropyl methylcellulose, USP (Methocel | 28.60 |
| | E6 LVP) | |
| 4 | Simethicone Emulsion Solids, USP (30% w/w | 2.08 |
| | Emulsion) | |
| 5 | Sodium lauryl sulfate, NF | 14.20 |
| 6 | Fenofibrate, USP Micronized | 130.00 |
| | Theoretical Capsule Weight | 272.88 |

The process of manufacturing the dosage form in accordance with the invention as follows (Example 1 & 2):
a) Sugar-coating is prepared by mixing sucrose, gum Acacia, Talc and Purified water to make a suspension and this suspension is than either sprayed onto the hallow capsules using spraying gun or poured to achieve the sugar coat onto the substrate. The process is continued the desired weight is achieved.
b) The drug suspension is prepared by first mixing Item #3 into item #2 until all of Item #3 is dissolved. The approximate mixing time is 60 minutes.
c) Add Item #4 & #5 while mixing and continue for mixing for not less than 15 minutes.
d) Add Item #6 while mixing and continue mixing for 30 minutes.
e) Homogenize the previous step for 15 minutes using a suitable homogenizer at a medium speed.
f) Continue mixing for not less than 15 minutes before starting the layering process.
g) Load the inactive substrate (Item #1) into the coating pan.
h) Begin layering with a sugar coating suspension prepared step (a) followed by drug suspension prepared in Step (f) using the following coating pan parameters:

| | | | |
|---|---|---|---|
| Inlet temperature: | 50-60 C. | Coating pan size: | 15″ |
| Inlet air volume: | 75 cfm | Coating pan speed: | 19 RPM |
| | | Baffle: | 2 |

The color suspension may be applied on the surface of the drug layer for ease of ink printing and to avoid the direct contact with the drug during the handling of the drug product.

An optional seal layering solution consisting of hydroxypropyl methylcellulose in water can be applied on the sugar coated substrate before spraying the drug layering suspension. The seal layering amount of 2-5% is preferred based on the starting weight of the inactive capsules.

An optional color layering suspension can be applied on as an outer layer to avoid the exposure of the drug while handling the drug product. The preferred coat amount is the range of 2-5%.

### Example 2

### Composition of a Capsule Dosage Form as Per the Present Invention

| Item # | Ingredients | Mg |
|---|---|---|
| 1 | Hollow substrate as per example 1 | 98.00 |

Drug layering formula

| | | |
|---|---|---|
| 6 | Purified water, USP | — |
| 7 | Hydroxypropyl methylcellulose, USP (Methocel | 3.25 |
| | E6 LVP) | |
| 8 | Simethicone Emulsion Solids, USP (30% w/w | 0.15 |
| | Emulsion) | |
| 9 | Doxycycline monohydrate, micronized | 10.00 |

Delayed-release coat formula

| | | |
|---|---|---|
| 10 | Eudragit L30D Solids (30% w/w dispersion) | 21.3 |
| 11 | Triethyl citrate | 4.26 |
| 12 | Purified Water | — |

Drug layering formula

| | | |
|---|---|---|
| 13 | Purified water, USP | — |
| 14 | Hydroxypropyl methylcellulose, USP (Methocel | 9.75 |
| | E6 LVP) | |
| 15 | Simethicone Emulsion Solids, USP (30% w/w | 0.45 |
| | Emulsion) | |
| 16 | Doxycycline monohydrate, micronized | 30.00 |

Seal-coating formula

| | | |
|---|---|---|
| 17 | Purified water, USP | — |
| 18 | Hydroxypropyl methylcellulose, USP (Methocel | 6.4 |
| | E6 LVP) | |
| | Theoretical Capsule Weight | 186.56 |

### Example 3

### Composition of a Capsule Dosage Form as Per the Present Invention

| Item # | Ingredients | Mg |
|---|---|---|
| 1 | Hollow substrate from Example 1 | 120.00 |

Drug layering formula

| | | |
|---|---|---|
| 6 | Purified water, USP | — |
| 7 | Hydroxypropyl methylcellulose, USP (Methocel | 10.00 |
| | E6 LVP) | |
| 8 | Simethicone Emulsion Solids, USP (30% w/w | 2.00 |
| | Emulsion) | |
| 9 | Omeprazole | 40.0 |

Buffered Seal-Coating formula

| | | |
|---|---|---|
| 10 | Purified water, USP | — |
| 11 | Hydroxypropyl methylcellulose, USP (Methocel | 6.0 |
| | E6 LVP) | |
| 12 | Calcium Carbonate powder | 300.0 |
| | Theoretical Capsule Weight | 554.00 |

### Example 4

### Composition of a Capsule Dosage Form as Per the Present Invention

| Item # | Ingredients | Mg |
|---|---|---|
| 1 | Hollow substrate from Example 1 | 98.00 |

Drug layering formula

| | | |
|---|---|---|
| 2 | Purified water, USP | — |
| 3 | Hydroxypropyl methylcellulose, USP (Methocel | 1.5 |
| | E6 LVP) | |
| 4 | Simethicone Emulsion Solids, USP (30% w/w | 0.20 |
| | Emulsion) | |
| 5 | Dutasteride powder | 0.5 |

Seal-coating formula

| | | |
|---|---|---|
| 6 | Purified water, USP | — |
| 7 | Hydroxypropyl methylcellulose, USP (Methocel | 6.4 |
| | E6 LVP) | |
| | Theoretical Capsule Weight | 71.6 |

### Example 5

### Composition of a Capsule Dosage Form as Per the Invention

| Item # | Ingredients | Mg |
|---|---|---|
| 1 | Hollow substrate as per example 1 | 101.00 |

Drug layering formula

| | | |
|---|---|---|
| 2 | Purified water, USP | — |
| 3 | Hydroxypropyl methylcellulose, USP (Methocel | 60.0 |
| | E6 LVP) | |
| 4 | Polyethylene glycol 400 | 10.0 |
| 5 | Simethicone Emulsion Solids, USP (30% w/w | 2.7 |
| | Emulsion) | |
| 6 | Celecoxib powder | 200.0 |

Seal-coating formula

| | | |
|---|---|---|
| 7 | Purified water, USP | — |
| 8 | Hydroxypropyl methylcellulose, USP (Methocel | 10.00 |
| | E6 LVP) | |
| | Theoretical Capsule Weight | 383.7 |

### Example 6

### Composition of a Capsule Dosage Form as Per the Invention

| Item # | Ingredients | Mg |
|---|---|---|
| 1 | Hollow substrate as per example 1 | 101.00 |

Drug layering formula

| | | |
|---|---|---|
| 2 | Purified water, USP | — |
| 3 | Hydroxypropyl methylcellulose, USP (Methocel | 60.0 |
| | E6 LVP) | |
| 4 | Simethicone Emulsion Solids, USP (30% w/w | 2.7 |
| | Emulsion) | |
| 5 | Thalidomide | 200.0 |

Seal-coating formula

| | | |
|---|---|---|
| 6 | Purified water, USP | — |
| 7 | Hydroxypropyl methylcellulose, USP (Methocel | 15.0 |
| | E6 LVP) | |
| | Theoretical Capsule Weight | 340.7 |

We claim:

1. A pharmaceutical composition in unit dose form comprising: a hollow substrate and one or more coatings on the hollow substrate, wherein at least one coating comprises at least one active pharmaceutical ingredient.

2. The pharmaceutical composition of claim 1, further comprising at least one additional coating between the hollow substrate and the at least one coating comprising the at least one active pharmaceutical ingredient.

3. The pharmaceutical composition of claim 2, wherein the at least one additional coating is selected from the group consisting of immediate release coatings, protective coatings, enteric or delayed release coatings, sustained release coatings, barrier coatings, seal coatings, and combinations thereof.

4. The pharmaceutical composition of claim 1, further comprising at least one top coating on the at least one coating comprising the at least one active pharmaceutical ingredient.

5. The pharmaceutical composition of claim 4, wherein the at least one top coating is selected from the group consisting of immediate release coatings, protective coatings, enteric or delayed release coatings, sustained release coatings, barrier coatings, seal coatings, and combinations thereof.

6. The pharmaceutical composition of claim 1, further comprising at least one barrier coating between the hollow substrate and the at least one coating comprising the at least one active pharmaceutical ingredient, and at least one top coating selected from the group consisting of enteric or delayed release coatings, protective coatings, or combinations thereof, on the at least one coating comprising the at least one active pharmaceutical ingredient.

7. The pharmaceutical composition of claim 1, wherein the active pharmaceutical ingredient is selected from the group consisting of analgesics, anti-inflammatory agents, anti-helminthics, anti-arrhythmic agents, anti-asthma agents, anti-bacterial agents, anti-viral agents, anti-coagulants, anti-dementia agents, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, immunosuppressants, anti-protozoal agents, anti-thyroid agents, anti-tussives, anxiolytics, sedatives, hypnotics, neuroleptics, neuroprotective agents, β-blockers, cardic inotropic agents, cell adhesion inhibitors, corticosteroids, cytokine receptor activity modulators, diuretics, anti-parkinsonian agents, gastro-intestinal agents, histamine H-receptor antagonists, keratolytics, lipid regulating agents, muscle relaxants, nitrates and other anti-anginal agents, non-steroid anti-asthma agents, nutritional agents, opioid analgesics, sex hormones, stimulants and anti-erectile dysfunction agents, and combinations thereof.

8. The pharmaceutical composition of claim 7, wherein the active pharmaceutical ingredient is selected from the group consisting of fenofibrate, doxycycline, omeprazole, dutasteride, celecoxib, thalidomide, and salts thereof.

9. The pharmaceutical composition of claim 1, wherein the dosage form is suitable for oral administration.

10. The pharmaceutical composition of claim 1, wherein the hollow substrate is hard or soft capsule.

11. The pharmaceutical composition of claim 1, wherein the at least one coating comprising the at least one active pharmaceutical ingredient is applied by spray coating.

12. A method of treating, preventing or reducing the occurrence of a condition in a subject, comprising administering to the subject the pharmaceutical composition of claim 1.

13. The method of claim 12, wherein the condition is selected from the group consisting of: gastrointestinal conditions, cardiovascular conditions, and pain-related conditions.
